(19) United States
(12) Patent Application Publication
(10) Pub. No.: US 2022/0105310 A1
(43) Pub. Date: Apr. 7, 2022

(54) HOLLOW SHAFT AND CATHETER

(71) Applicant: ASAHI INTECC CO., LTD., Seto-shi (JP)

(72) Inventor: Masaru YAGI, Seto-shi (JP)

(73) Assignee: ASAHI INTECC CO., LTD., Seto-shi (JP)

(21) Appl. No.: 17/552,055

(22) Filed: Dec. 15, 2021

Related U.S. Application Data
(63) Continuation of application No. PCT/JP2020/020318, filed on May 22, 2020.

(30) Foreign Application Priority Data
Jun. 21, 2019 (JP) 2019-115323

(51) Int. Cl. A61M 25/00

(52) U.S. Cl. CPC A61M 25/005; A61M 25/0026

(57) ABSTRACT

A hollow shaft and a catheter that, with a braided body, reduce a compressive force toward a second lumen positioned between a first lumen and a shaft outer peripheral surface even when subjected to an external force, and suppress the collapse of the second lumen. The catheter includes a shaft having a first lumen and a second lumen extending in the longitudinal axis direction, and a braided body extending in the longitudinal axis direction of the shaft and disposed inside the shaft, the braided body enclosing both the first lumen and the second lumen. In a transverse cross-sectional direction of the shaft, an inner peripheral surface of the shaft, which defines the first lumen, has a protruding surface protruding toward the center of the shaft, and the second lumen is located between the protruding surface and an outer peripheral surface of the shaft.

### CROSS REFERENCE TO RELATED APPLICATIONS

This application is a continuation application of International Application No. PCT/JP2020/020318, filed May 22, 2020, which claims priority to Japanese Patent Application No. 2019-115323, filed Jun. 21, 2019. The contents of these applications are incorporated herein by reference in their entirety.

### TECHNICAL FIELD

This application relates to a hollow shaft and a catheter.

### BACKGROUND

As a catheter to be inserted into a blood vessel, there has been proposed a catheter provided with a tube having a plurality of lumens (for example, see Japanese Unexamined Patent Application Publication No. 2013-509941).

The tube of the catheter disclosed in Japanese Unexamined Patent Application Publication No. 2013-509941 includes a main lumen having a substantially cross-shaped cross-section, and a plurality of supplementary lumens disposed around the main lumen. As a result of the main lumen having a substantially cross-shaped cross-section, the tube has a tendency to collapse in a specific direction under an external force.

However, in the tube described above, when the tube is subjected to an external force, the main lumen collapses in a certain direction and the supplementary lumens also collapse.

### SUMMARY

An object of the disclosed embodiments is to provide a hollow shaft and a catheter that, with a braided body, are capable of reducing a compressive force toward a second lumen positioned between a first lumen and a shaft outer peripheral surface even when subjected to an external force, and are capable of suppressing the collapse of the second lumen.

In order to achieve the above object, a hollow shaft, which is an aspect of the disclosed embodiments, includes: a shaft having a first lumen and a second lumen extending in a long axis direction; and a braided body extending in the long axis direction of the shaft and disposed so as to enclose both the first lumen and the second lumen inside the shaft; wherein in a transverse cross-section of the shaft, an inner peripheral surface of the shaft, which defines the first lumen, has a protruding surface protruding toward the center of the shaft, and the second lumen is located between the protruding surface and an outer peripheral surface of the shaft.

The shaft may have a plurality of second lumens, the inner peripheral surface may have a plurality of protruding surfaces, and each of the second lumens may be located between one of the protruding surfaces and the outer peripheral surface of the shaft.

A first tubular body extending in the long axis direction of the shaft may be disposed inside the first lumen, and an outer peripheral surface of the first tubular body may be joined with the inner peripheral surface that defines the first lumen, and/or a second tubular body extending in the long axis direction of the shaft may be disposed inside the second lumen, and an outer peripheral surface of the second tubular body may be joined with an inner peripheral surface that defines the second lumen.

In a transverse cross-section of the hollow shaft, the braided body may be disposed along an outer edge of the first lumen and an outer edge of the second lumen of the shaft.

In a transverse cross-section of the hollow shaft, the braided body may be disposed along an outer edge of the first tubular body and an outer edge of the second tubular body of the shaft.

A catheter, which is an aspect of the disclosed embodiments, may comprise the hollow shaft described above.

### BRIEF DESCRIPTION OF THE DRAWINGS

FIG. 1 is a perspective view of a hollow shaft according to a first embodiment.

FIG. 2 is a transverse sectional view of the hollow shaft according to the first embodiment.

FIG. 3 is a transverse sectional view of a hollow shaft according to a second embodiment.

FIG. 4 is an overall view of a catheter.

FIG. 5 is a longitudinal sectional view of a catheter shaft and a tip in portion A of FIG. 4.

FIG. 6 is a transverse sectional view of a hollow shaft according to a modification.

FIG. 7 is a transverse sectional view of a hollow shaft according to a modification.

### DETAILED DESCRIPTION

Hereinafter, hollow shafts according to disclosed embodiments will be described with reference to the drawings. The disclosure is not intended to be limited to the specific embodiments described in the drawings.

### First Embodiment

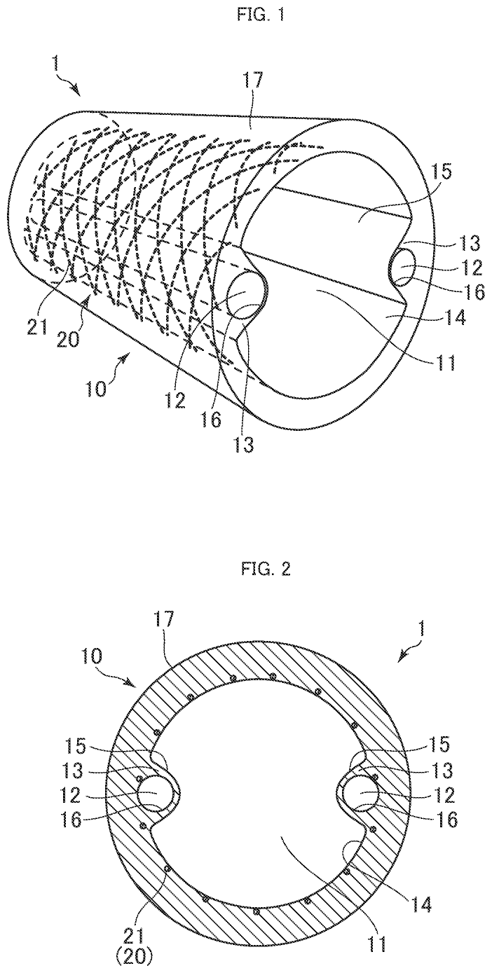

FIG. 1 is a perspective view of a hollow shaft 1 according to a first embodiment.

FIG. 2 is a transverse sectional view of the hollow shaft 1 according to the first embodiment.

As shown in FIG. 1, the hollow shaft 1 includes a shaft 10 and a braided body 20.

The shaft 10 has a substantially cylindrical shape, and has a first lumen 11 and a plurality of second lumens 12 (e.g., two in this embodiment). The shaft 10 comprises a plurality of convex portions 13 (e.g., two in this embodiment) protruding toward the center and extending along the long axis direction of the shaft 10 on the inner peripheral side. The first lumen 11 extends along the long axis direction of the shaft 10, and is defined by a first inner peripheral surface 14. The first inner peripheral surface 14 has protruding surfaces 15 configuring the surfaces of the convex portions 13. That is to say, the protruding surface 15 protrudes toward the center of the shaft 10.

Each of the second lumens 12 has a cylindrical shape extending along the long axis direction of the shaft 10, and is defined by a second inner peripheral surface 16. In the radial direction of the shaft 10, each of the second lumens 12 is located between one of the protruding surfaces 15 and an outer peripheral surface 17 of the shaft 10. At least a part of each second lumens 12 is located in each of the convex portions 13.

The material constituting the shaft 10 is not particularly limited as long as it does not impair the effects of the disclosed embodiments. For example, when the shaft 10 is used as a medical device which is inserted inside the body, it preferably has antithrombogenicity, flexibility, and biocompatibility, and as examples of the material, a resin material such as a polyamide resin, a polyolefin resin, a polyester resin, a polyurethane resin, a silicone resin, and a fluororesin can be adopted. The material may be changed in the long axis direction of the shaft 10. For example, a part of the shaft 10 may be made of a softer material (a material with lower bending stiffness) than the other part.

In terms of the dimensions of the shaft 10, for example, the outer diameter is 1.5 to 5 mm, the inner diameter (maximum width) of the first lumen 11 is 1.3 to 4.5 mm, the inner diameter of the second lumens 12 is 0.2 to 0.6 mm, the wall thickness of the shaft 10 is 0.2 to 0.6 mm, and the wall thickness between the first lumen 11 and the second lumens 12 of the shaft 10 is 0.02 to 0.10 mm.

The braided body 20 is formed by weaving a plurality of wires 21 in a lattice pattern to form a tube shape as a whole. The braided body 20 is embedded inside the side surface of the shaft 10 and extends in the long axis direction of the shaft 10, and is disposed so as to enclose both the first lumen 11 and the plurality of second lumens 12 inside the shaft 10. As shown in FIG. 2, in a transverse cross-section of the hollow shaft 1, the braided body 20 is disposed along the outer edge of the first lumen 11 and the outer edges of the second lumens 12 of the shaft 10. That is to say, a part of the braided body 20 is disposed adjacent to the first lumen 11 and the second lumens 12.

A single wire or a plurality of wires can be used as each of the wires 21 constituting the braided body 20, and each of the wires 21 constituting the braided body 20 may be formed, for example, from a twisted wire in which a plurality of metal wires having different wire diameters or the like are twisted together.

A metallic material or a resin material can be used as the material forming the wires 21 of the braided body 20. Examples of the resin material include polyamide, polyester, polyarylate, and polyether ether ketone. From the viewpoint of improving the strength and flexibility, it is preferable to use a metallic material. Examples of the metallic material include stainless steel such as SUS304, a nickel-titanium alloy, a cobalt-chromium alloy, and tungsten steel. Each of the wires 21 may be formed of the same material, or may be formed of different materials.

Furthermore, from the viewpoint of improving the visibility of the braided body 20, the material constituting the wires 21 of the braided body 20 may be a radiopaque material. Examples of the radiopaque material include gold, platinum, tungsten, and alloys containing these elements (such as a platinum-nickel alloy). The radiopaque material may be a combination of such a radiopaque material and another material, such as a material which is coated on the surface of a non-radiopaque material.

The hollow shaft 1 described above includes a shaft 10 having a first lumen 11 and second lumens 12 extending in the long axis direction, and a braided body 20 extending in the long axis direction of the shaft 10 and disposed so as to enclose both the first lumen 11 and the second lumens 12 inside the shaft 10. In a transverse cross-section of the shaft 10, a first inner peripheral surface 14 of the shaft 10, which defines the first lumen 11, has protruding surfaces 15 protruding toward the center of the shaft 10, and the second lumens 12 are located between the protruding surfaces 15 and an outer peripheral surface 17 of the shaft.

According to the hollow shaft 1, because the second lumen 12 is located between the protruding surface 15 and the outer peripheral surface 17 of the shaft even when subjected to an external force, the second lumen 12 is less susceptible to an external force applied in the radial direction of the hollow shaft 1, and further, because the braided body 20 enables the compressive force toward the second lumen 12 to be reduced, the collapse of the second lumen 12 can be suppressed. In addition, the braided body 20 also enables the collapse of the first lumen 11 to be suppressed.

The shaft 10 has a plurality of second lumens 12, and the first inner peripheral surface 14 has a plurality of protruding surfaces 15. Each of the second lumens 12 is located between one of the protruding surfaces 15 and the outer peripheral surface 17 of the shaft 10. Even when there are a plurality of second lumens 12 as described above, because each of the second lumens 12 is located between the protruding surface 15 and the outer peripheral surface 17 of the shaft, the second lumens 12 are less susceptible to an external force applied in the radial direction of the hollow shaft 1, and further, because the braided body 20 enables the compressive force toward the second lumens 12 to be reduced, the collapse of the second lumens 12 can be suppressed.

In a transverse cross-section of the hollow shaft 1, the braided body 20 is disposed along the outer edge of the first lumen 11 and the outer edges of the second lumens 12 of the shaft 10. In this way, the braided body 20 is disposed along the outer edges of the first lumen 11 and the second lumens 12, which facilitates the formation of a smaller outer diameter of the hollow shaft 1.

### Second Embodiment

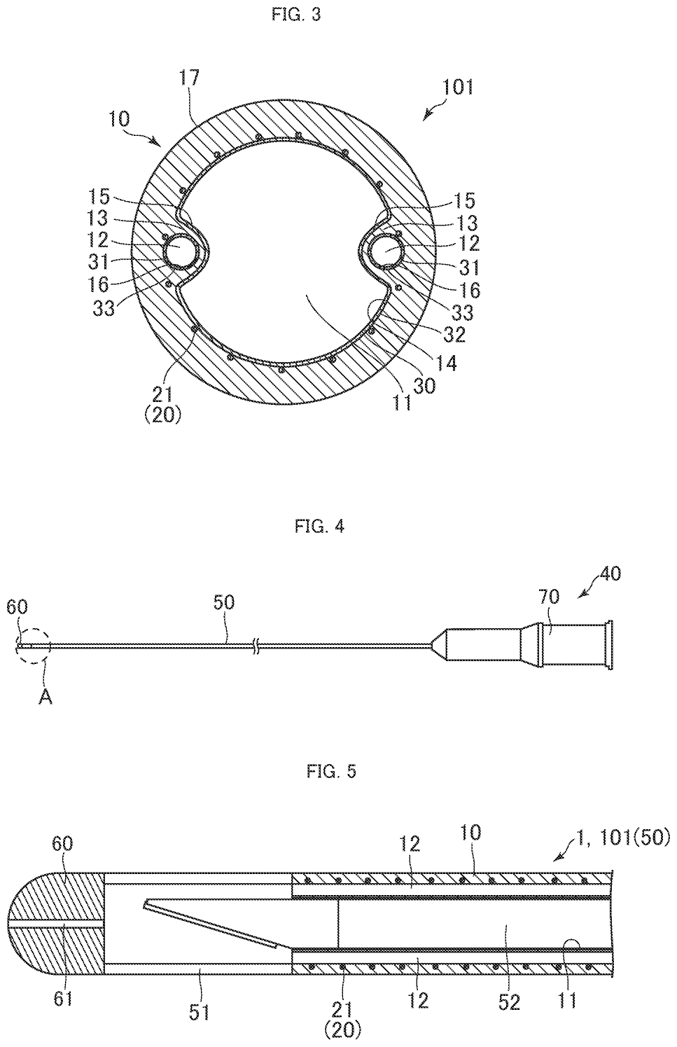

Next, a hollow shaft 101 according to a second embodiment will be described with reference to FIG. 3. Note that, in the hollow shaft 101 of this embodiment, the same members as in the hollow shaft 1 of the first embodiment are given the same reference numerals, and a detailed description is omitted.

FIG. 3 is a transverse sectional view of the hollow shaft 101 according to the second embodiment.

The hollow shaft 101 includes a shaft 10, a braided body 20, a first tubular body 30, and a plurality of second tubular bodies 31.

The first tubular body 30 has a tubular shape extending in the long axis direction of the shaft 10, and is disposed inside the first lumen 11. The outer shape of the first tubular body 30 is a shape that matches that of the first lumen 11. In a transverse cross-section of the hollow shaft 101, an outer peripheral surface 32 of the first tubular body 30 makes contact with, and is joined to, the first inner peripheral surface 14 of the first lumen 11.

Each second tubular body 31 has a tubular shape extending in the long axis direction of the shaft 10, and is disposed inside the second lumen 12. The outer shape of the second tubular body 31 is a shape that matches that of the second lumen 12. In a transverse cross-section of the hollow shaft 101, an outer peripheral surface 33 of each second tubular body 31 makes contact with, and is joined to, the second inner peripheral surface 16 of the second lumen 12.

In a transverse cross-section of the hollow shaft 101, the braided body 20 is disposed along the outer edge of the first tubular body 30 and the outer edge of the second tubular body 31 of the shaft 10. That is to say, a part of the braided body 20 is disposed so as to make contact with the outer edge of the first tubular body 30 and the second tubular body 31.

As the material constituting the first tubular body 30 and the second tubular body 31, for example, a resin material such as a polyamide resin, a polyolefin resin, a polyester resin, a polyurethane resin, a silicone resin, or a fluororesin can be used. In terms of the dimensions of the first tubular body 30, for example, the wall thickness is 0.01 to 0.05 mm. Note that, by appropriately selecting the material constituting of the first tubular body 30 and the second tubular body 31 and wall thickness of the first tubular body 30 and the second tubular body 31, the bending rigidity may be made higher than the bending rigidity of the shaft 10.

According to the hollow shaft 101 of this embodiment, the first tubular body 30 extending in the long axis direction of the shaft 10 is disposed inside the first lumen 11, and the outer peripheral surface 32 of the first tubular body 30 is joined with the first inner peripheral surface 14 that defines the first lumen 11. Each of the second tubular bodies 31 extending in the long axis direction of the shaft 10 is disposed inside the second lumen 12, and the outer peripheral surfaces 33 of second tubular body 31 are joined with the second inner peripheral surface 16 that defines the second lumen 12.

As a result of disposing the first tubular body 30 in the first lumen 11 and each of the second tubular bodies 31 in the second lumen 12 in this manner, it is possible to further suppress the collapse of the first lumen 11 and each of the second lumens under an external force.

In a transverse cross-section of the hollow shaft 101, the braided body 20 is disposed along the outer edge of the first tubular body 30 and the outer edge of the second tubular body 31 of the shaft 10. In this way, the braided body 20 is disposed along the outer edges of the first tubular body 30 and the second tubular body 31, which facilitates the formation of a smaller outer diameter of the hollow shaft 1. Furthermore, when the hollow shaft according to this embodiment is produced, the second tubular body 31 is disposed so as to be fitted in the protruding surface 15 (outer surface of the protruding portion of the first tubular body 30), and is secured to the first tubular body 30 by the braided body 20. As a result of welding the shaft 10 in a state where the first tubular body 30 and the second tubular body 31 are secured in this manner, the production can be performed without shifting of the relative positions of the first tubular body 30 and the second tubular body 31 during welding of the shaft 10.

Next, a catheter 40 will be described as an example in which the hollow shaft 1 or 101 described above is applied.

FIG. 4 is a schematic view of the catheter 40.

The catheter 40 is, for example, an ultrasound imaging catheter. The catheter 40 mainly includes a catheter shaft 50, a tip 60 joined to the distal end of the catheter shaft 50, and a connector 70 joined to the proximal end of the catheter shaft 50. Further, the hollow shaft 1 or 101 described above is used on the distal side of the catheter shaft 50.

FIG. 5 is a longitudinal sectional view of the catheter shaft 50 and the tip 60 in part A of FIG. 4.

As shown in FIG. 5, in the distal end side of the hollow shaft 1 or 101, a window portion 51 having a cylindrical shape and being made of an ultrasound-permeable material is provided. At the distal end of the window portion 51, the tip 60 inside which a through hole 61 is formed is provided. A rotating imaging core 52 whose distal end is equipped with an ultrasound transducer is accommodated inside the first lumen 11. For example, an operating wire inserted from the proximal end of the hollow shaft 1 or 101 and inserted through to the distal end portion of the hollow shaft 1 or 101 is inserted into a second lumen 12, and the angle of the distal end of the hollow shaft 1 or 101 is manipulated with respect to the central axis of the hollow shaft 1 or 101 using the operating wire.

In the catheter 40, an electrical signal from an ultrasound diagnostic imaging device (not shown) is converted into an ultrasound wave in the ultrasound transducer and irradiated with respect to an observation target site, and a reflected wave from the tissue is once again converted into an electrical signal and transmitted to the ultrasound diagnostic imaging device (not shown). As a result, the observation target site is visualized in the ultrasound diagnostic imaging device (not shown).

According to the catheter 40 described above, by providing the hollow shaft 1 or 101, it is possible to prevent a device inserted into the first lumen 11 from becoming stuck due to the collapse of the first lumen 11, to prevent a drug or the like flowing through the second lumen 12 from not reaching a treatment site due to the collapse of the second lumen 12, and to prevent the sliding resistance of an inserted device such as an operating wire inserted through the second lumen 12 from increasing due to the collapse of the second lumen 12.

The embodiment in which the hollow shaft 1 or 101 is applied is not limited to the catheter 40, and it may, for example, be applied to a catheter or a balloon catheter used to diagnose or treat a constricted portion or an occluded portion.

Note that the disclosure is not intended to be limited to the configurations of the above-described embodiments. It will be appreciated that the above-disclosed features and functions, or alternatives thereof, may be desirably combined into different methods and devices. Also, various alternatives, modifications, variations or improvements may be subsequently made by those skilled in the art, and are also intended to be encompassed by the disclosed embodiments. As such, various changes may be made without departing from the spirit and scope of this disclosure.

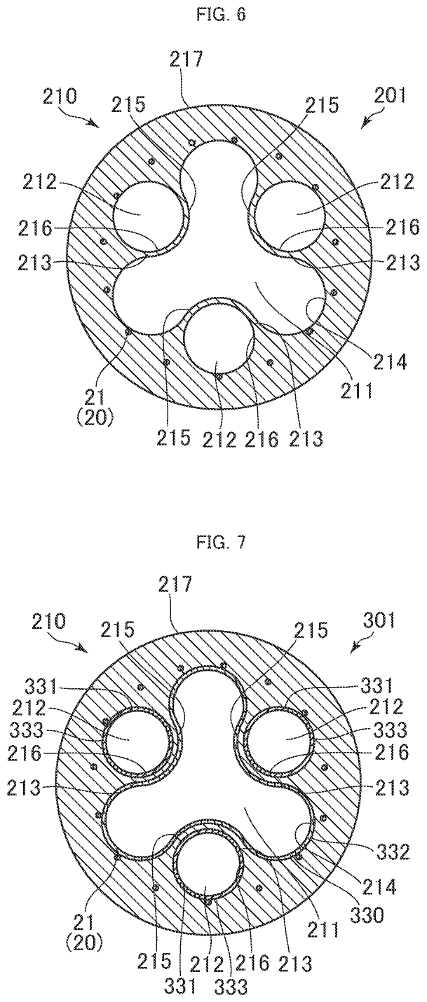

For example, as illustrated by the hollow shaft 201 in FIG. 6, a shaft 210 may be provided with three convex portions 213 on the inner peripheral side so as to make a first lumen 211 have a substantially three-pronged shape in a transverse cross-section. Therefore, the first inner peripheral surface 214 has three protruding surfaces 215 constituting the surfaces of the three convex portions 213. Further, in the radial direction of the shaft 210, three second lumens 212 defined by second inner peripheral surfaces 216 are each located between one of the protruding surfaces 215 and an outer peripheral surface 217 of the shaft 210. The wires 21 of the braided body 20 are disposed along the outer edge of the first lumen 211 and the outer edge of the second lumen 212 of the shaft 210.

As illustrated by the hollow shaft 301 in FIG. 7, a first tubular body 330 may be disposed inside the first lumen 211 of the hollow shaft 201 of FIG. 6, and a second tubular body 331 may be disposed inside each second lumen 212. An outer peripheral surface 332 of the first tubular body 330 makes contact with, and is joined to, the first inner peripheral surface 214 of the first lumen 211, and an outer peripheral surface 333 of each second tubular body 331 makes contact with, and is joined to, the second inner peripheral surface 216 of a second lumen 212. The wires 21 of the braided body 20 are disposed along the outer edge of the first tubular body 330 and the outer edge of the second tubular body 331 of the shaft 210.

The number of protruding surface 15 or 215 and the number of second lumen 12 or 212 are not limited to the number in the embodiments described above, and may be one or more. Although the number of protruding surface 15 or 215 and the number of second lumen 12 or 212 are the same, the number of second lumen 12 or 212 may be less than the number of protruding surface 15 or 215.

In the hollow shaft 101 or 301, both the first tubular body 30 or 330 and the second tubular body 31 or 331 are provided in the first lumen 11 or 211 and the second lumen 12 or 212, but only one of these may be provided.

The first inner peripheral surface 14 or 214 may be curved or formed from straight lines and vertices in a transverse cross-section of the hollow shaft 1, 101, 201 or 301, that is to say, may have a substantially triangular or rectangular shape.

The second lumen 12 or 212 may be circular, or may have a substantially triangular or rectangular shape in a transverse cross-section of the hollow shafts 1, 101, 201 or 301.

The protruding surface 15 or 215 may have a shape which is curved toward the center of the hollow shaft 1, 101, 201 or 301, or formed from straight lines and vertices in a transverse cross-section of the hollow shaft 1, 101, 201 or 301, that is to say, may have a substantially triangular or rectangular shape.

What is claimed is:

1. A hollow shaft comprising:
a shaft having a first lumen defined by an inner peripheral surface of the shaft and at least one second lumen, the first lumen and the at least one second lumen extending along a longitudinal axis direction of the shaft;
and a braided body extending in the longitudinal axis direction and disposed inside the shaft, the braided body enclosing the first lumen and the at least one second lumen,
wherein, in a transverse cross-sectional direction of the shaft, the inner peripheral surface of the shaft has at least one protruding surface protruding toward a center of the shaft, and
the at least one second lumen is located between the at least one protruding surface and an outer peripheral surface of the shaft.

2. The hollow shaft according to claim 1, wherein the shaft has a plurality of second lumens,
the inner peripheral surface has a plurality of the protruding surfaces, and
each of the second lumens is located between each of the protruding surfaces and the outer peripheral surface of the shaft.

3. The hollow shaft according to claim 1, wherein at least one of:
(i) a first tubular body extending in the longitudinal axis direction of the shaft is disposed inside the first lumen, and an outer peripheral surface of the first tubular body is joined with the inner peripheral surface that defines the first lumen, and
(ii) a second tubular body extending in the longitudinal axis direction of the shaft is disposed inside the second lumen, and an outer peripheral surface of the second tubular body is joined with an inner peripheral surface that defines the second lumen.

4. The hollow shaft according to claim 1, wherein, in the transverse cross-sectional direction of the hollow shaft, the braided body is disposed along an outer edge of the first lumen and an outer edge of the second lumen of the shaft.

5. The hollow shaft according to claim 3, wherein, in the transverse cross-sectional direction of the hollow shaft, the braided body is disposed along at least one of an outer edge of the first tubular body and an outer edge of the second tubular body of the shaft.

6. A catheter comprising a hollow shaft according to claim 1.
